(19) United States
(12) Patent Application Publication
(10) Pub. No.: US 2021/0153896 A1
(43) Pub. Date: May 27, 2021

(54) Device for Intraosseous Needle Removal

(71) Applicants: ESCUELA UNIVERSITARIA DE ENFERMERIA Y FISIOTERAPA SAN JUAN DE DIOS. ORDEN HOSPITALARIA DE DE SAN JU, Madrid (ES); UNIVERSIDAD PONTIFICIA COMILLAS, Madrid (ES)

(72) Inventor: Julio Cesar DE LA TORRE MONTERO, Madrid (ES)

(21) Appl. No.: 17/257,147

(22) PCT Filed: Jul. 9, 2019
(86) PCT No.: PCT/ES2019/070479

§ 371 Date: Dec. 30, 2020

(30) Foreign Application Priority Data
Jul. 10, 2018 (ES) 201830688

(51) Int. Cl. A61B 17/34

(52) U.S. Cl. CPC A61B 17/3472; A61B 2017/00367; A61B 17/3496

(57) ABSTRACT

The present invention concerns, but is not limited to, a new medical device suitable for the removal of an intraosseous needle implanted in a bone of a patient. The device comprises a traction handle (3) formed by a gripping part (4) coupled to a shank (5), which are disposed in a substantially T-shaped arrangement. In addition, a distal end of the shank (5) comprises a connector (6) complementary to a connector disposed on the needle (1). In this way, an ergonomic device is obtained for generating the traction and rotation forces necessary to remove the intraosseous needle (1) from the patient's bone. The device also contains biosafety elements for carrying out this removal technique, such as a protective sleeve (54) for protecting against accidental punctures and a protector (55) that protects the patient's skin surface during the removal process.

### FIELD OF THE INVENTION

The present invention is comprised in the technical field corresponding to the medical device sector. More specifically, the invention concerns, but is not limited to, a device specially designed for the process for the safe removal of intraosseous needles.

### BACKGROUND OF THE INVENTION

When administering or drawing fluids to/from a patient through the intravascular route, whether for medicinal products or blood transfusions, there are two alternatives: administration through the intravenous route or through the intraosseous route. In certain situations, where bone marrow is required to be removed, where the patient has sustained burns, presents edemas, or in emergency situations in patients requiring cardiopulmonary resuscitation, the intraosseous route is a preferred solution for the delivery of liquid solutions to the patient through the intravascular route. In the last mentioned, for use in emergencies, it is key to use elements that protect both the patient and the health care staff carrying out the removal technique, which must be safe.

Once said administration of fluid has been performed, it is necessary to remove the needle from the body of the patient. However, intraosseous injection and/or administration devices are generally not adapted for facilitating the method of removal, which complicates said method and can generate damage, discomfort, or pain for the patient, in addition to being harmful for the medical professional carrying out the removal technique, as they are not used in usual clinical practice, nor are they described in protocols for the removal of tools suitable for that purpose, but rather the removal is performed with an instrument designed for another purpose.

To mitigate the aforementioned problem, there are some products on the market, such as, for example, the “Arrow EZ-IO Needle Set” marketed by the company Teleflex Inc. Said device is based on a syringe with a connection to an intraosseous needle. Once the syringe is connected to the needle, the medical personnel proceeds to manual removal by means of a joint rotation and traction movement on the syringe to remove the implanted needle. A non-specialized device, such as a syringe, is thereby used by way of a handle or gripping part. Given the geometry of the needle and the nature of the forces applied during removal, that is, rotation and traction, maintaining alignment between the needle and the syringe is a complex task which may cause damage to tissues near the needle insertion point or a feeling of discomfort in the patient. Moreover, and given the design of intraosseous needles, the use of a device not specifically designed for the safe removal presents a risk to anti-accidental puncture safety for the professional performing the technique.

Needle insertion devices such as the one described in U.S. Pat. No. 5,868,711, which discloses an implantable intraosseous needle assembly and various means for injecting, using, and extracting that assembly into a bone containing bone marrow are also known.

Said document also relates to an extractor comprising an outer positioning element, by way of a sleeve, wherein the intraosseous needle is partially protected in the interior once it has been removed, minimizing the risks of accidental punctures. Protection, however, is not complete, as the distal portion which includes the tip of the needle in the mentioned device continues to protrude through the through hole of its receiver, even once it has been assembled in the conical standoff acting as a connector therein. Therefore, the sleeve disclosed in the extractor assembly of U.S. Pat. No. 5,868,711 is not a protective element, per se, but rather an element for positioning the extractor with respect to the base thereof. Furthermore, the removal operation with this device is preferably performed by means of a successive rotation mechanism, which allows the intraosseous needle to be removed by means of turning a proximal handle. This mechanism therefore causes it to be necessary to rotate the needle multiple times while it is still within the bone or tissue for complete removal, which may damage said bone or tissue during the removal process.

As described in patent application US2010152616, there is also known in the state of the art a device for safely accessing bone marrow and other tissues. Said device comprises a needle assembly, a sensor mechanism, and an actuator for the insertion of the needle in the tissue of a patient. The device also comprises a needle protector which is configured for sliding inside the base when the needles are inserted in the skin, by way of a guide. In this context, said document refers exclusively to devices for the insertion and not the removal of needles, therefore being relative to a technical problem different from the one of the present application. The present invention proposes an ergonomic device for the removal of intraosseous needles, specially adapted for exerting the traction and rotation forces necessary to remove said needles in a simple and minimally aggressive manner for the patient, in addition to providing safety for the professional carrying out the safe removal technique, when performing removal of the device with a complete and specific biosafety element.

### BRIEF DESCRIPTION OF THE INVENTION

The technical problem solved by the present invention with respect to the prior art mentioned in the preceding section is to provide improved protection features for the user of the device during the operation of removal of the intraosseous needle. This is because given that once the needle has been removed, or even at virtually the same time, depending on the skill of the user, the user may immediately and readily extend a protective element concentric with the needle, along the entire length thereof, preventing any potential damage with the pointed end of said needle. It is further mentioned that in the arrangement of the present invention, easier removal is observed as a rotational movement can be combined with a traction movement, which is not found in the aforementioned prior art documents. The user could perform said traction movement simultaneously with a movement for extending the protective element, thereby combining an enhanced protection for the user handling the device with a faster and cleaner removal operation that is, therefore, less painful for the patient.

Likewise, the present invention provides a device for the removal of needles wherein the of removal operations can be performed by the user by means of a single traction movement, accompanied by a gentle rotation, but without the need for multiple rotations of the device for removing the needle, as occurs with the device disclosed in U.S. Pat. No. 5,868,711. The risk of injury in the tissue of the patient during the removal process is therefore significantly reduced.

More specifically, the object of the present invention generally concerns a device for the removal of an intraosseous needle, said needle preferably being of the type comprising a Luer-Lock connector, and comprising a traction handle for the removal of the device from the body of a patient.

Throughout the present document, the expression “traction handle” will be used to designate any control mechanism adapted or configured for removing the intraosseous needle by means of the user performing a traction action, such that said traction causes the device for removal to move in the direction substantially parallel to the axis of the intraosseous needle, moving it away from the body of the patient.

Advantageously, said traction handle comprises a gripping part and a shank disposed in a substantially T-shaped arrangement, wherein a distal end of said shank comprises a connector complementary to the Luer-Lock connector arranged in the intraosseous needle.

It is thereby achieved that the method for the removal of the needle is simpler simple for health care professionals, providing an ergonomic device specially designed for exerting rotation and traction forces, but always controlled by the user of the device. Hence, during removal of the needle, the alignment of said needle with the device for removal is facilitated, such that oscillations of the needle are minimized and less damage therefore occurs in the tissues and the bone of the patient.

In a preferred embodiment of the invention, the complementary connector is a Luer-Lock threaded hole.

In a preferred embodiment of the invention, the material of the device comprises polypropylene.

The invention is therefore provided made of a rigid material capable of withstanding the required torsional stresses.

In a preferred embodiment of the invention, the end of the shank opposite the handle comprises an extendible protective sleeve substantially concentric with the complementary connector housed in the shank.

Risks in the case of accidental punctures of medical staff with the needle once it has been removed are minimized by means of this design.

In a preferred embodiment of the invention, the distal end of the shank comprises a surface adapted for being placed on the skin surface of the body of a patient without causing local damage or rashes in the area surrounding the puncture point. It is an elastic element that conserves the integrity of the patient's skin without causing accidental rashes.

Better support of the device and better alignment of the shank with the needle is enabled as a result.

Likewise, and preferably, the shank of the device comprises at least one side wall, an inner cavity concentric to said wall, and a longitudinal opening from the inner cavity through the side wall. It is thereby possible to act on the protective sleeve from outside the device, in a safe manner and minimizing the risk of contamination or puncture of the user during the removal operation.

More particularly, the device comprises a slide-type button mechanically linked to the protective sleeve, so that it can be manipulated from outside the device.

According to another aspect of the invention, the protective sleeve comprises a housing for an elastic element at its end distal to the gripping part. A damping feature relating to the contact of the protective sleeve and further minimization of the risk of sustaining damage are thereby offered.

Additionally, the protective sleeve comprises a first fixing element and the slide button comprises a second fixing element cooperating with the first fixing element, such that the slide-type button is fixed to the protective sleeve. It is through said slide button that the protective sleeve can be operated, extending it or retracting it, although once the needle has been removed, the protective sleeve will be locked in its fully extended state, to prevent accidental punctures.

In a preferred embodiment of the invention, the slide-type button comprises a ridged area so that it can be gripped better by the hand and fingers of health care professionals handling the device, preferably by pushing it with the thumb.

More particularly, the ridged area is located on the upper face of the slide button for easier operation and extension or retraction of the protective sleeve, depending on whether or not the device is going to be used.

According to another aspect of the invention, the longitudinal opening comprises a widening for the insertion of the slide button so that it can be assembled and fixed on the protective sleeve, specifically, and preferably the first fixing element with the second fixing element.

According to another aspect of the invention, the longitudinal opening comprises a first groove element intended for facilitating and orienting the sliding of the slide button.

Complementarily, the slide-type button comprises a second groove element, wherein the first groove element cooperates with said second groove element such that the slide-type button can slide along the longitudinal opening of the shank and the protective sleeve can slide along the longitudinal inner cavity.

According to a first function of the invention, the protective sleeve can move from a first retracted position, in which the protective sleeve is substantially concealed in the longitudinal inner cavity, to a second extended position, in which the protective sleeve covers the intraosseous needle.

According to a second function of the invention, the protective sleeve can move from a second extended position, in which the protective sleeve covers the intraosseous needle, to a first retracted position, in which the protective sleeve is substantially concealed in the longitudinal inner cavity.

### DESCRIPTION OF THE FIGURES

FIG. 1 shows a device for the removal of an intraosseous needle, as well as said intraosseous needle, according to a preferred embodiment of the present invention.

FIG. 2A shows a respectively exploded perspective view of a device for the removal of an intraosseous needle according to a preferred embodiment of the present invention.

FIG. 2B shows a perspective view of a device for the removal of an intraosseous needle, where the protective sleeve has a first retracted position, according to a preferred embodiment of the present invention.

FIG. 2C shows a perspective view of a device for the removal of an intraosseous needle, where the protective sleeve has a second extended position, according to a preferred embodiment of the present invention.

FIG. 3A specifically shows a plan view of a device for the removal of an intraosseous needle according to a preferred embodiment of the present invention.

FIG. 3B specifically shows a profile view of a device for the removal of an intraosseous needle according to a preferred embodiment of the present invention.

FIG. 3C specifically shows a cross-section view of a device for the removal of an intraosseous needle according to a preferred embodiment of the present invention.

### REFERENCE NUMBERS USED IN THE FIGURES

For the purpose of helping to better understand the technical features of the invention, the mentioned figures are accompanied by a series of reference numbers, where the following has been depicted in an illustrative and non-limiting manner:
(1) intraosseous needle
(2) Luer-Lock connector
(3) traction handle
(4) gripping part
(5) shank
(51) side wall
(52) inner cavity
(53) opening
(53a) widening
(53b) first groove element
(54) protective sleeve
(54a) first fixing element
(54b) housing
(55 elastic element
(56) slide button
(56a) second fixing element
(56b) second groove element
(56c) ridged area
(56d) upper face
(6) complementary connector
(7) surface at the distal end of the shank
(81) first retracted position
(82) second extended position

### DETAILED DESCRIPTION OF THE INVENTION

A preferred embodiment of the present invention, provided for illustrative but non-limiting purposes thereof, is described below.

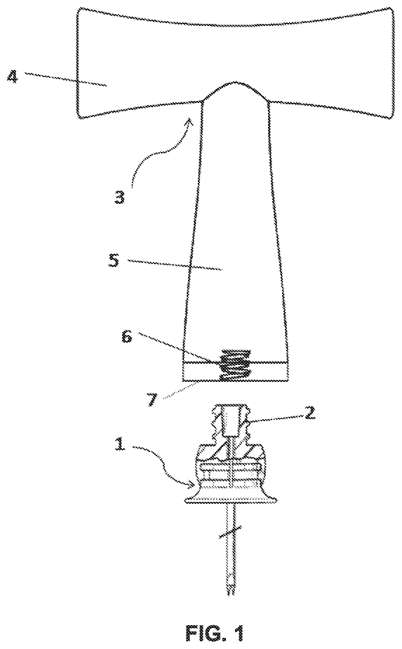

The main object of the invention concerns, as shown in FIG. 1 and based on that described in the preceding sections, a specialized device for the method of removing an intraosseous needle (1) implanted in a bone of a patient. Generally, for the definition of the invention, intraosseous needles (1) known in the state of the art, which comprise a Luer-Lock type connector (2) disposed on the needle (1), shall be taken into consideration.

In turn, the device for removal comprises a traction handle (3), as defined above in the present invention, wherein said traction handle (3) comprises a gripping part (4) coupled to a shank (5) disposed in a substantially T-shaped arrangement. Likewise, the distal end of the shank (5) comprises a connector (6) complementary to the Luer-Lock connector (2) disposed on the intraosseous needle (1), such that the forces exercised during removal are transmitted by means of this connection.

By means of this configuration of the traction handle (3), where the shank (5) is centered and substantially perpendicular to the gripping part (4), an ergonomic device specially designed for generating rotation and traction forces for the purpose of removing the intraosseous needle (1) can be provided. The alignment of the needle (1) with the traction handle (3) is therefore facilitated during said removal, such that the needle (1) is prevented from bending and damaging both tissues and bone close to the area of insertion of the intraosseous needle (1).

For removal of the inserted intraosseous needle (1), the shank (5) of the device is positioned substantially aligned with the needle (1) such that the Luer-Lock connector (2) of the needle (1) can be coupled with the complementary connector (6) arranged on the shank (5). In this way, once the shank (5) is correctly aligned and the device is connected to the needle (1), when rotation and traction forces are exercised on the traction handle (3), removal of said needle (1) is achieved, preventing bending therein and, hence, minimizing damage in tissues near the area of insertion of the needle (1).

The device for removal must be able to withstand the traction and torsional stresses to which the device is subjected during removal. In a preferred embodiment of the invention, the device for the removal of intraosseous needles (1) is made up of polypropylene.

In another even more preferred embodiment of the invention, both the Luer-Lock connector (2) and the complementary connector (6) arranged on the shank (5) of the device are threaded connectors.

Additionally, the invention has protective measures for the use of sharp health care instruments (biosafety elements in work equipment for accidents with a biological risk) against possible undesired punctures that may occur during the method of removal of the inserted intraosseous needle (1). To that end, in a preferred embodiment, the invention comprises a protective sleeve (54) arranged on the distal end of the shank (5) which is deployed at the same time the needle (1) is removed. The intraosseous needle (1) is thereby protected and contained inside the protective sleeve (54) once it has been removed, minimizing the risks of accidental punctures. As such, the invention is also, but not exclusively, comprised as a biosafety product as provided under the definitions adopted by the CDC (USA), FDA (USA), NIOSH (USA), OSHA (USA), GERES (France), and SIROH (Italy), those health care apparatus, instruments, or materials incorporating protective safety systems and designed for the purpose of eliminating or minimizing risks of exposure to accidental injuries and contagion derived from the use of sharp objects are known as biosafety devices.

The object of the invention would be comprised within an active safety device where, depending on the arrangement and design of the protective sleeve, it can be used with semi-automatic activation elements which raise the safety to passive, that is, without the need for intervention of the professional.

In another preferred embodiment, the distal surface (7) of the shank (5) is adapted for being placed on the body of the patient. Stability during the movements performed during the removal of the needle (1) is thereby favored, preventing said intraosseous needle (1) from bending and minimizing damage to surrounding tissues.

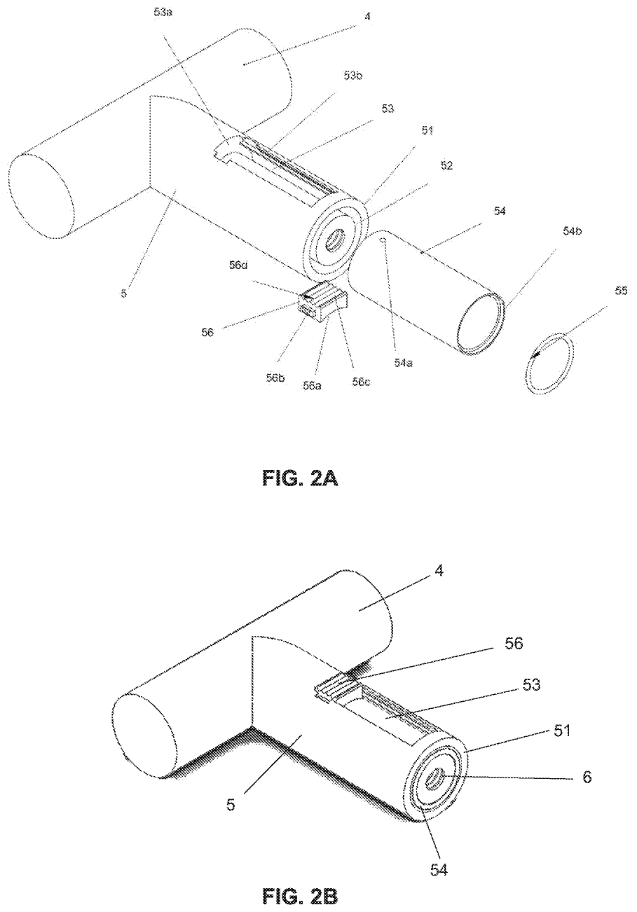

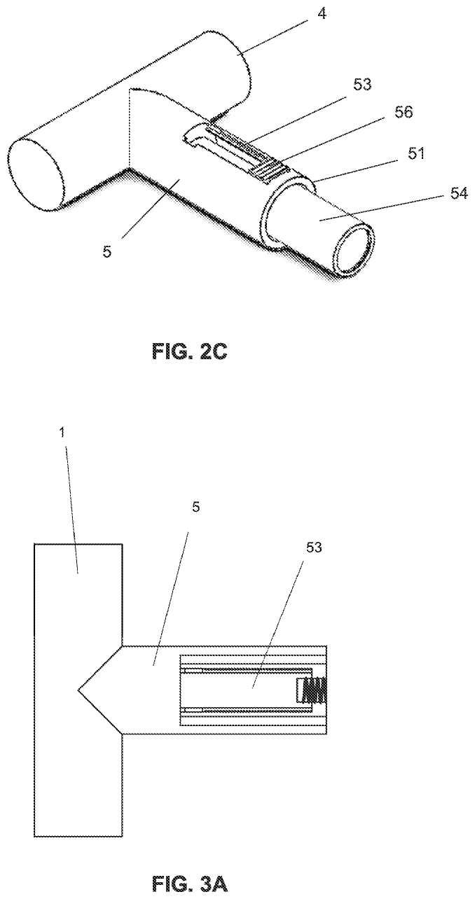

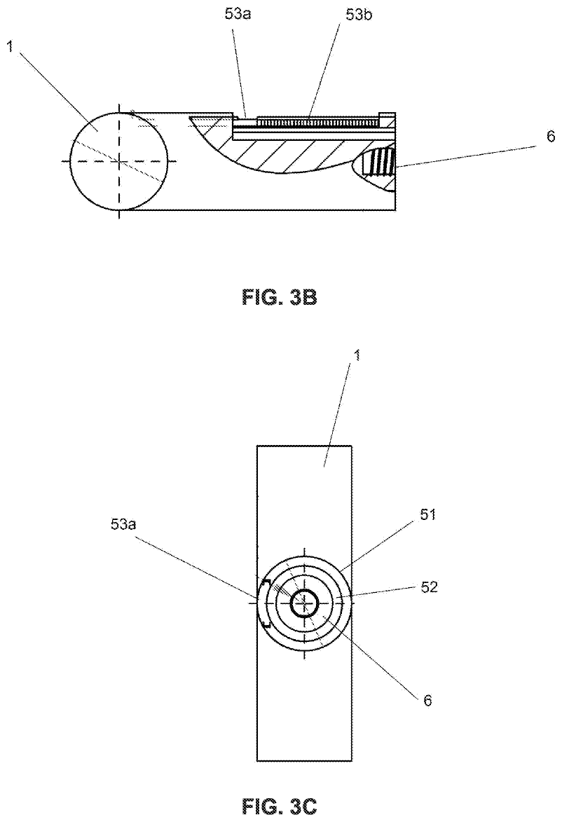

Thus and also as observed in FIG. 2A et seq., the device for the removal of an intraosseous needle (1), said needle (1) being of the type comprising a Luer-Lock connector (2), comprises a traction handle (3), said traction handle (3) comprises a gripping part (4) and a shank (5) disposed in a substantially T-shaped arrangement, where a distal end of said shank (5) comprises a connector (6) complementary to the Luer-Lock connector (2) arranged in the intraosseous needle (1), where the end of the shank (5) opposite the handle (3) comprises a protective sleeve (54) extendible from a longitudinal inner cavity (52) in the shank (5), wherein the protective sleeve (54) is substantially concentric with the connector (6) complementary housed in the shank (5).

Moreover, the shank (5) comprises at least one side wall (51), a concentric inner cavity (52), and a longitudinal opening (53) from the longitudinal inner cavity (52) through the side wall (51).

More particularly, the device comprises a slide-type button (56) mechanically linked to the protective sleeve (54).

Additionally, the protective sleeve (54) comprises a housing (54b) for an elastic element (55) at its end distal to the gripping part (4). Said elastic element (55) is preferably a rubber washer.

According to another aspect of the invention, the protective sleeve (54) comprises a first fixing element (54a) and the slide-type button (56) comprises a second fixing element (56a) cooperating with the first fixing element (54a), such that the slide-type button (56) is fixed to the protective sleeve (54). In a preferred manner, the first fixing element (54a) is a hole, and the second fixing element (56a) is a protrusion.

More particularly, the slide-type button (56) comprises a ridged area (56c).

In a particular embodiment, the ridged area (56c) is located on the upper face (56d) of the slide button (56).

More specifically, the longitudinal opening (53) comprises a widening (53a) for the insertion of the slide button (56).

Moreover, the longitudinal opening (53) comprises a first groove element (53b).

According to a preferred embodiment of the invention, the slide-type button (56) comprises a second groove element (56b), wherein the first groove element (53b) cooperates with said second groove element (56b) such that the slide-type button (56) can slide along the longitudinal opening (53) of the shank (5) and the protective sleeve (54) can slide along the longitudinal inner cavity (52).

According to a first function of the device of the invention, the protective sleeve (54) can move from a first retracted position (81), in which the protective sleeve (54) is substantially concealed in the longitudinal inner cavity (52), to a second extended position (82), in which the protective sleeve (54) covers the intraosseous needle (1).

According to a second function of the device of the invention, the protective sleeve (54) can move from a second extended position (82), in which the protective sleeve (54) covers the intraosseous needle (1), to a first retracted position (81), in which the protective sleeve (54) is substantially concealed in the longitudinal inner cavity (52).

1. A device for the removal of an intraosseous needle, said needle being of the type comprising a Luer-Lock connector, where the device comprises a traction handle, and said traction handle comprises a gripping part and a shank disposed in a substantially T-shaped arrangement, wherein a distal end of said shank comprises a connector complementary to the Luer-Lock connector disposed in the intraosseous needle,
wherein the distal end of the shank opposite the handle comprises a protective sleeve, extendible from a longitudinal inner cavity in the shank, wherein the protective sleeve is substantially concentric with the complementary connector housed in the shank.

2. The device according to claim 1, wherein the complementary connector comprises a Luer-Lock threaded hole.

3. The device according to claim 1, wherein the material of the device comprises polypropylene.

4. The device according to claim 1, wherein the distal end of the shank comprises a surface adapted for being placed on the body of a patient.

5. The device according to claim 1, wherein the shank comprises at least one side wall, a concentric inner cavity and a longitudinal opening from the longitudinal inner cavity through the side wall.

6. The device according to claim 5, comprising a slide-type button mechanically adapted for the deployment and the withdrawal of the protective sleeve.

7. The device according to claim 6, wherein the protective sleeve comprises a housing for an elastic element at its end distal to the gripping part.

8. The device according to claim 6, wherein the protective sleeve comprises a first fixing element and the slide-type button comprises a second fixing element cooperating with the first fixing element, such that the slide-type button is fixed to the protective sleeve.

9. The device according to claim 8, wherein the slide-type button comprises a ridged area.

10. The device according to claim 9, wherein the ridged area is located on the upper face of the slide button.

11. The device according to claim 8, wherein the longitudinal opening comprises a widening for the insertion of the slide button.

12. The device according to claim 9, wherein the longitudinal opening comprises a first groove element.

13. The device according to claim 12, wherein the slide-type button comprises a second groove element, wherein the first groove element cooperates with said second groove element such that the slide-type button can slide along the longitudinal opening of the shank and the protective sleeve can slide along the longitudinal inner cavity.

14. The device according to claim 5, wherein the protective sleeve can move from a first retracted position, in which the protective sleeve is substantially concealed in the longitudinal inner cavity, to a second extended position, in which the protective sleeve covers the intraosseous needle.

15. The device according to claim 5, wherein the protective sleeve can move from a second extended position, in which the protective sleeve covers the intraosseous needle, to a first retracted position, in which the protective sleeve is substantially concealed in the longitudinal inner cavity.
